SEQUENCE LISTING (8 sequences)

SEQ ID NO: 1 - protein, 657 aa, Artificial
  Synthetic
AAKPLDKSSSTLHHGHSNTQIPYTITVNGTSQNILSSLTFNKNQNISYKDIENKVKSVLYFNRGISDIDLRLSKQAEYTVHFKNGTKRVIDLKSGTYTADLINTSDIKAISVNVDTKKQPKDKAKANVQVPYTITVNGTSQNILSNLTFNKNQNISYKDLEDRVKSVLESNRGITDVDLRLSKQAKYTVNFKNGTKKVIDLKAGIYTANLINSSDIKSININVDTKKHIENKAKRNYQVPYSINLNGTSTNILSNLSFSNKPWTNYKNLTSQIKSVLKHDRGISEQDLKYAKKAYYTVYFKNGGKRILQLNSKNYTANLVHVKDVKRIEITVKTGTKAKADRYVPYTIAVNGTSTPILSDLKFTGDPRVGYKDITKKVKSVLKHDRGIGERELKYAKKATYTVHFKNGKKKVINLNSKISQLNLLYVQDIKKIDIDVKTGSKAKADSYVPYTIAVNGTSTPILSKLKISNKQLISYKYLNDKVKSVLKNERGISDLDLKFAKQAKYTVYFKNGKKQVVNLKSDIFTPNLFSAKDIKKIDIDVKTGSKAKADSYVPYTIAVNGTSTPILSKLKISNKQLISYKYLNDKVKSVLKSERGISDLHLKFAKQAKYTVYFKNGKKQVVNLKSDIFTPNLFSAKDIKKIDIDVKQYTKSKKNK

SEQ ID NO: 2 - protein, 10 aa, Artificial
  Synthetic
AAKQIDKSSS

SEQ ID NO: 3 - protein, 8 aa, Artificial
  Synthetic
KVDIEIKF

SEQ ID NO: 4 - protein, 9 aa, Artificial
  Synthetic
QYTKSKKNK

SEQ ID NO: 5 - protein, 10 aa, Artificial
  Synthetic
AAKQIDKSSS

SEQ ID NO: 6 - protein, 8 aa, Artificial
  Synthetic
KVDIEIKF

SEQ ID NO: 7 - protein, 10 aa, Artificial
  Synthetic
AAKPLDKSSS

SEQ ID NO: 8 - protein, 7 aa, Artificial
  Synthetic
TKSKKNK

(19) United States
(12) Patent Application Publication
(10) Pub. No.: US 2008/0234189 A1
(43) Pub. Date: Sep. 25, 2008

(54) USE OF A COMPOSITION

(76) Inventors: Jan-Ingmar FLOCK, Bromma (SE); Triantafyllos Chavakis, Heideberg (DE); Mathias Herrmann, Saarbrucken (DE); Klaus T. Preissner, Giessen (DE)

Correspondence Address: BROWDY AND NEIMARK, P.L.L.C.;624 NINTH STREET, NW, SUITE 300, WASHINGTON, DC, 20001-5303, US

(21) Appl. No.: 11/865,279

(22) Filed: Oct. 1, 2007

Related U.S. Application Data
(62) Division of application No. 10/298,206, filed on Nov. 18, 2002, now abandoned.
(60) Provisional application No. 60/331,782, filed on Nov. 21, 2001.

(30) Foreign Application Priority Data
Nov. 16, 2001 (SE) SE0103831-4

(51) Int. Cl. A61K 38/16; A61P 19/02; A61P 11/06; A61P 17/06

(52) U.S. Cl. 514/12

(57) ABSTRACT

A method of treatment of a mammal suffering from an inflammatory condition or a cancer includes administering an anti-inflammatory protein or a peptide fraction of the protein having at least one repeating unit, the protein being selected from a group of proteins designated Eap (Extracellular adherence protein). The inflammatory condition may be a non-bacterial or bacterial inflammation.

### TECHNICAL FIELD OF THE INVENTION

The present invention relates to the use of a protein or a peptide portion thereof, said protein being selected from a group of proteins designated Eap (extracellular adherence protein). More specifically it relates to the therapeutic use of said protein or polypeptide in the treatment of acute and chronic inflammatory responses and in the treatment of cancer.

### BACKGROUND OF THE INVENTION

The inflammatory response is a defence reaction caused by tissue damage or injury. This may result from a variety of causes, both bacterial infections and physical and chemical factors, such as heat, ionising radiation, toxic substances, mechanical factors etc. Examples of such tissue damage or injury are abrasions, broken bones, muscle and tendon strains, sprains, joint dislocations, sunburns, fire burns etc. The inflammatory response also may be related to and aggravate e.g. states of allergy, such as hay fever, bee sting, as well as autoimmune diseases, such as asthma, arthritis, Crohn's disease etc.

Since inflammation is a defence mechanism of the body in response to tissue injury or damage or as a reaction to immunological activation, the primary objective thereof is to localize and reduce or eliminate the irritant and repair the surrounding tissue. Due to different causes, an inflammatory response may be triggered by release of inflammatory compounds from various sources such as injured tissue cells, lymphocytes and mast cells into the extracellular fluid, the most important being histamine, prostaglandins, and cytokines.

The triggered inflammatory response involves three major stages: dilation of capillaries to increase blood flow; microvascular permeability changes and escape of plasma proteins from the bloodstream; and leukocyte recruitment including adhesion and transmigration through endothelium and accumulation at the site of injury. In the last stage, the leukocyte accumulation at the site of injury is the result of the so-called leukocyte adhesion cascade, which is a sequence of adhesion and activation steps involving different adhesion receptors (such as selectins and integrins) on leukocytes. Those steps may be identified as capture, rolling, slow rolling, firm adhesion and transmigration. Each step in the leukocyte adhesion cascade is necessary for effective leukocyte recruitment into the site of inflammation, and blocking any of them would lead to a reduction of leukocyte accumulation in the tissue. Regardless of its origin, the inflammation is characterized by a number of symptoms, viz. redness, swelling, heat, pain and loss of tissue or organ function. The inflammatory condition may be of varying severity, ranging from scarcely noticeable to severely disabling, and may even, in extreme cases, be life-threatening.

Various anti-inflammatory drugs are currently used to combat disabling or dangerous states of inflammation, based on the physiological mechanisms of the inflammatory response. They function as blockers, suppressors, or modulators thereof. Essentially, they may be subdivided into two major groups: steroidal and nonsteroidal (NSAID) agents. Both types of agents have well-known side effects, although these are generally less severe for the NSAIDs.

For example, topically applied steroids have side effects such as dry, irritated skin, and unusual growth of hair on the face or body after prolonged use. The application of potent corticosteroids to extensive areas of the body for prolonged periods increases the likelihood of systemic side effects, whereas common side effects associated with oral steroids include diarrhoea or constipation, headache, nervousness, just to mention a few. Other administration forms, such as inhalation, are associated with still other side effects. The side effects of NSAIDs are generally less severe, however, these latter anti-inflammatory drugs are less potent. Moreover, long-term or extensive ingestion of NSAIDs can result in toxic effects for the kidney or the stomach epithelium, possibly causing ulcers.

It therefore appears that there is a continuing need of providing new anti-inflammatory drugs for use in methods of anti-inflammatory treatment.

### SUMMARY OF THE INVENTION

According to a first aspect of the present invention new anti-inflammatory drugs are provided by use of a protein or of a peptide portion thereof comprising at least one repeating unit of said protein, said protein being selected from a group of proteins designated Eap (extracellular adherence protein).

According to a second aspect, the present invention provides a method of treating a mammal suffering from an inflammatory condition.

According to a further aspect of the invention the above defined protein or polypeptide is used in the manufacture of a medicament for use in a cancer therapy.

Further aspects of the invention are defined in the claims.

### BRIEF DESCRIPTION OF THE DRAWINGS

FIG. 1 is a bar diagram illustrating experimental data relating to the in vivo inhibition of neutrophil emigration by Eap in acute inflammation in mice;

FIG. 2A is a bar diagram illustrating experimental data relating to the contribution of Eap to the adhesion of S. aureus to ICAM-1; and

FIG. 2B is a graph illustrating experimental data relating to the contribution of Eap to the adhesion of S. aureus to ICAM-1.

### DETAILED DESCRIPTION OF THE INVENTION

The present invention in a first aspect relates to the inhibition of inflammatory reactions in a mammal, such as a human, by administration of a certain protein, selected from the Eap group (Extracellular adherence protein), or a suitable peptide portion of said protein. This aspect of the invention is based on the surprising discovery that proteins belonging to the Eap group, or a suitable peptide portion thereof, present anti-1-inflammatory effects when given to a mammal suffering from an acute or chronic inflammation.

It has been shown that proteins belonging to the Eap group are produced by the bacterium Staphylococcus aureus. S. aureus is a persistent pathogen that causes serious community-acquired and nosocomial infections. The range of disease produced by S. aureus is broad, including endocarditis, osteomyelites and septic shock. Eap has a wide binding repertoire; it has affinity for at least seven plasma proteins, including fibrinogen, fibronectin and prothrombin. The protein also has an ability to bind to cells of S. aureus, to form oligomers and to agglutinate S. aureus.

The Eap group of proteins should be considered as a family of proteins, here termed the Eap-family, or just Eap, since minor variations in sequence occur between different strains of S. aureus. It comprises an extracellular 60 kDa protein secreted by the bacterium (1). This family also comprises a protein designated Map (Major histocompatibility complex class II Analogous Protein) (2) (3). Another member of the family is a cationic protein termed p70 (4, 5). In a recent study, Eap was found to be present in 98% of clinical isolates from 240 strains of S. aureus. Table 1 identifies proteins within the Eap family and illustrates the relationship between them.

TABLE 1
C-terminal and N-terminal sequences of proteins belonging to the Eap group

| | Source of | N-terminal | | | | | | |
|---|---|---|---|---|---|---|---|---|
| | the protein; | amino | C-terminal | | Accession | Version | | |
| Pro- | bacterial | acid se- | amino | Data- | Number | Number | | |
| tein | strain | quence | acid sequence | base | (AC) | (SV) | Date | Ref. |
| Eap | S. aureus, | N-AAKQIDKSSS | KVDIEIKF-C | Gen- | AJ132841 | AJ132841.1 | Mar. 25, 1999 | (8) |
| | Newman | | | Bank | | GI:4454323 | | |
| Map | S. aureus, | Not determined | QYTKSKKNK-C | Gen- | AJ223806 | AJ223806.1 | Jan 7, 1999 | (6) |
| | Newman | | | Bank | | GI:4138455 | | |
| Map | S. aureus, | N-AAKQIDKSSS | KVDIEIKF-C | Gen- | U20503 | U20503.1 | Sep. 30, 1995 | (3) |
| | FDA 574 | | | Bank | | GI:1001960 | | |
| P70 | S. aureus, | N-AAKPLDKSSS | TKSKKNK-C | Gen- | Y10419 | Y10419.1 | Jun. 8, 1997 | (7) |
| | wood 46 | | | Bank | | GI:2190506 | | |

It appears that the proteins partly differ in their terminal sequences; the proteins are more or less isoforms. A typical amino acid sequence example of a protein of the Eap group or protein family is the following (8):

AAKPLDKSSSTLHHGHSNTQI [symbol] [symbol] [symbol] [symbol]
IENKVKSVLYFNRGISDIDLRLSKQAEYTVHFKNGTKRVIDLKSGTYTAD
LINTSDIKAISVNVDTKKQPKDKAKANVQV [symbol] [symbol] [symbol] [symbol]
LEDRVKSVLESNRGITDVDLRLSKQAKYTVNFKNGTK
KVIDLKAGIYTANLINSSDIKSININVDTKKHIENKAKRNYQVPYSINLN
GTSTNILSNLSFSNKPWTNYKNLTSQIKSVLKHDRGISEQDLKYAKKAYY
TVYFKNGGKRILQLNSKNYTANLVHVKDVKRIEIT [symbol] [symbol] [symbol] [symbol]
FTGDPRVGYKDITKKVKSVLKHDRGIG
ERELKYAKKATYTVHFKNGKKKVINLNSKISQLNLLYVQDIKKIDID [symbol] [symbol] [symbol] [symbol] [symbol] [symbol] [symbol]
YLNDKVKSV
LKNERGISDLDLKFAKQAKYTVYFKNGKKQVVNLKSDIFTPNLFSAKDIK
KIDID [symbol] [symbol] [symbol] [symbol] ISNKQLISYKYLND
KVKSVLKSERGISDLHLKFAKQAKYTVYFKNGKKQVVNLKSDIFTPNLFS
AKDIKKIDIDVKQYTKSKKNK

Another similar sequence has been described (3). When used herein below, the term Eap will be understood to be any of the proteins within the Eap family. Eap has several repeating units of about 30 amino acids or more, which may differ in a few amino acids between different members of the family. For example, in the above sequence, several repeating units may be identified, such as e.g. PYTITVNGTSQNILSSLTFNKNQNISYK or VKTGTKAKADRYVPYTIAVNGTSTPILSDLK, with only one or two amino acids varying. Examples of repeating units are highlighted in bold characters, and a partial overlap of both repeating units with each other can be recognized as well. It is likely that several of the described characteristics of Eap can be found within a single repeating unit. Therefore, what is said herein about Eap is valid also for peptide portions of shorter length but comprising at least one repeating unit thereof. In other words, a peptide fraction of Eap suitable for use according to the invention should comprise at least one repeating unit of the amino acid sequence of the protein. For the purpose of the invention the word peptide fraction or peptide portion or peptide is used as synonymous with polypeptide.

As briefly outlined herein above, in relation to the inflammatory response, leukocytes emigrating from the blood-stream into sites of inflammation or injury, undergo a complex sequence of adhesion and locomotion steps. These highly co-ordinated processes require the expression and upregulation of various adhesion receptors on the surface of leukocytes and vascular cells. Different receptor systems direct the interaction of leukocytes with the endothelium. Whereas leukocyte rolling depends on selectins, firm adhesion to and transmigration through the endothelium is mediated by the β2-integrins Mac-1 (CD11b/CD18, αMβ2, CR3) and LFA-1 (CD11α/CD18, αLβ2), that interact with their counter-receptor ICAM-1 on the endothelial cells.

The present inventors have investigated whether Eap by binding to the different proteins of the extracellular matrix could regulate the adhesion and recruitment of leukocytes. The results indicate that the secreted bacterial protein Eap specifically interacts with ICAM-1 on endothelial cells, thereby inhibiting Mac-1 and LFA-1 mediated leukocyte adhesion to endothelial cells.

Thus, the present inventors have found that Eap binding to host (adhesive) proteins in the connective tissue (extracellular matrix, ECM) and on cell surfaces leads to inhibition of host (inflammatory) cell adhesion and migration and thereby blocks inflammatory defence mechanisms of the infected host organism. This anti-adhesive function of Eap was established for different types of leukocyte cells including granulocytes and monocytes, but can also be extended to lymphocytes, which all share several adhesive processes and characteristics including the existence of β2-integrins, the major class of adhesion receptors. Thus, by binding to different ligands of β2-integrins in the ECM (such as fibrinogen or vitronectin) and on cells (such as ICAM-1), Eap can inhibit the mobility, infiltration and activities of acute inflammatory cells (granulocytes), of monocytes and macrophages (relevant for phagocytosis) and of immune cells (such as lymphocytes).

By experiments as detailed in the experimental section herein below, the present inventors subsequently were able to show that Eap in vivo inhibits recruitment of neutrophils into a site of inflammation. It is on the basis of these findings that the invention has been made.

### EXPERIMENTAL

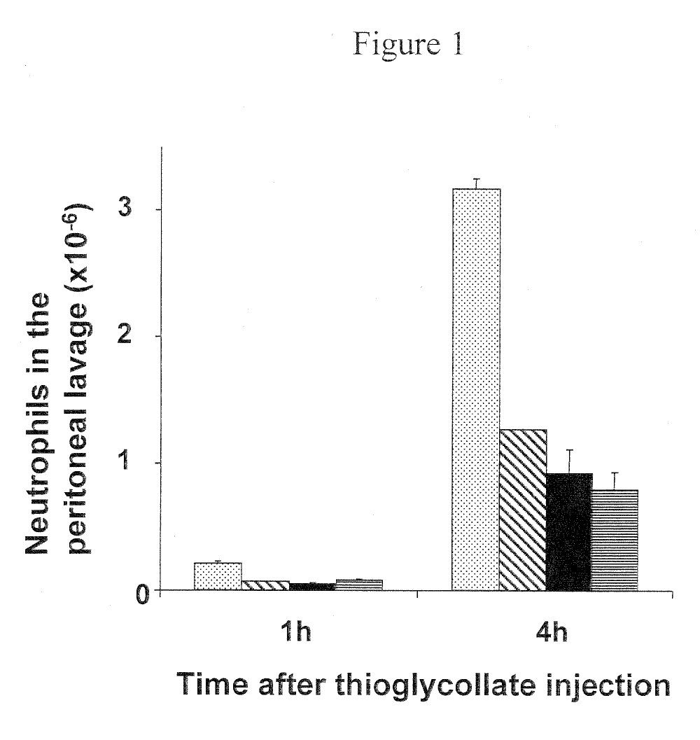

### 1. Inhibition of Neutrophil Recruitment in the Mouse Model of Acute Thioglycollate-Induced Peritonitis by Eap (FIG. 1)

### Methods:

Bacterial strains and purification of Eap. Previously, we have characterized the polymorphism of S. aureus type strains and clinical isolates (8) of which three different S. aureus strains were used in this study: Strain Newman D2C (ATCC 25904) is a laboratory strain rich in Clf, strain Wood 46 (ATCC 10832) is rich in protein A and S. aureus clinical isolate 7 from a patient with S. aureus soft tissue infection have been characterized as producer of a representative group of Eap. Eap of these strains, namely Eap N, Eap W and Eap 7, respectively, were purified by affinity chromatography on FBG-Sepharose followed by ion-exchange chromatography using a MonoS column (Pharmacia, Uppsala, Sweden) as described before (1). Moreover, Eap N, Eap W and Eap 7 were also recombinantly expressed in E. coli and isolated on Ni-NTA column. Bacteria were propagated in appropriate standard media (tryptic soy, brain heart infusion, Muller-Hinton, or Luria-Bertani)

In vivo peritonitis model: Experiments were performed according to a previously described protocol (9, 10), in which 1 ml thioglycollate bouillon (Merck, Darmstadt, Germany) was administered intraperitoneally to female 8-10-week old NMRI mice (Charles River Wiga, Sulzfeld, Germany) to induce peritonitis. For inhibition studies, 30 min prior to the injection of thioglycollate 100 μg of mAb against mouse Mac-1 or mouse LFA-1 in PBS or 50-100 μg of Eap in PBS were administered intravenously. Control mice were treated with the same volume of PBS and some mice obtained isotype-matched control antibodies. All reagents were endotoxin-free. At 1 h and 4 h after injection of thioglycollate, mice were sacrificed and the peritoneal lavage was generated by injecting 10 ml PBS, massaging the peritoneal wall and removing the fluid. Total cell numbers were determined in a Casy Counter (Schärfe System, Germany) and 5×10⁴ cells were then transferred onto adhesion slides (Biorad, Munich, Germany), fixed and stained (Diff-Quick, DADE-Behring, Munich, Germany). Cells were differentially counted by microscopy, evaluating 300 cells per slide. From the total cell count in the peritoneal lavage and the percentage of neutrophils determined microscopically, the absolute number of emigrated neutrophils in the peritoneal lavage was calculated. Analysis of blood smears revealed that peripheral neutrophil counts were not affected by any of the antibodies or reagents injected.

### Results:

Peritonitis was induced by thioglycollate injection, and after 4 h there was an expected increase in the total leukocyte count, mostly attributable to emigrated neutrophils: The percentage of neutrophils among all leukocytes after 4 h was 50-60% as compared with 3-10% 1 h after stimulation (9, 10). The use of blocking antibodies against LFA-1 or Mac-1 30 min prior to the induction of peritonitis resulted in a 50-75% inhibition of neutrophil extravasation into the inflamed peritoneum at 4 h following thioglycollate injection (FIG. 1), whereas isotype-matched control antibody had no effect at all (not shown). At 1 h and 4 h following thioglycollate injection neutrophil recruitment to the peritoneum was significantly reduced in mice that were pre-treated with Eap 7 (50, 75, 100 μg/mouse). The maximal inhibition (>75%) was obtained at 4 h with 100 μg of Eap. Thus, Eap inhibits β2-integrin-dependent neutrophil emigration in vivo.

The results are illustrated in FIG. 1, where:
dotted bars represent values obtained for mice treated with PBS prior to thioglycollate administration;
hatched bars represent values obtained for mice treated with a blocking mAb against mouse α-subunit of LFA-1 prior to thioglycollate administration;
filled bars represent values obtained for mice treated with a blocking mAb against mouse α-subunit of Mac-1 prior to thioglycollate administration; and
bars with horizontal lines represent values obtained for mice treated with Eap7 prior to thioglycollate administration.

Data are mean ±SEM (n=4 mice per treatment) of a typical experiment; similar results were obtained in three separate sets of experiments.

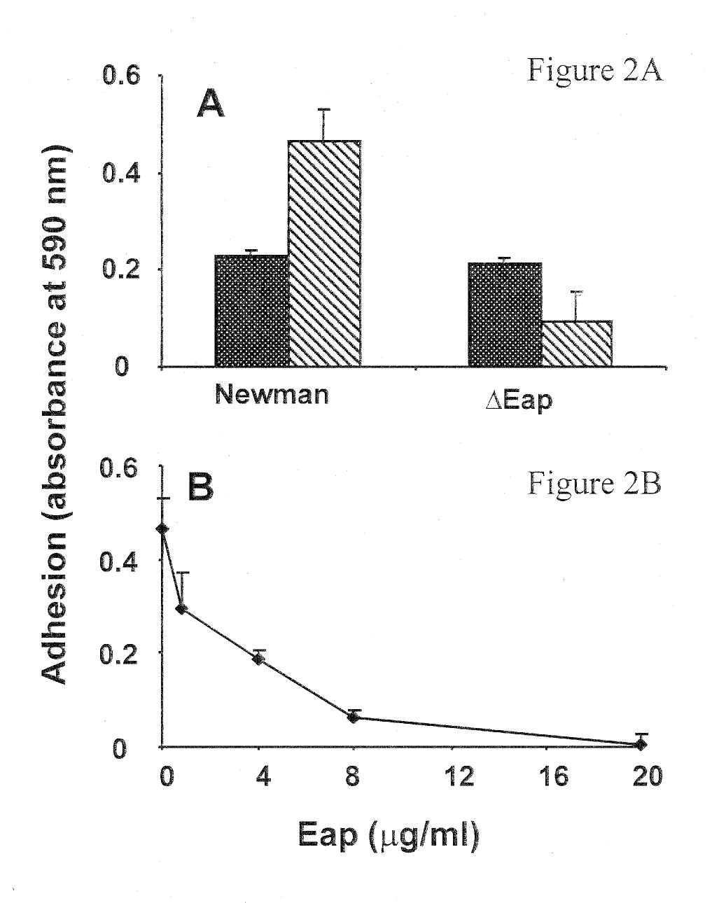

2. Interaction of Eap with Endothelial Cell ICAM-1 (FIG. 2)

### Methods:

Adherence of S. aureus: Polystyrene microtiter plate wells were coated with FBG or ICAM-1 (5 μg/ml each), respectively, dissolved in bicarbonate buffer, pH 9.6 and blocked with 3% (wt/vol) BSA. Formalin-inactivated S. aureus strain Newman or Eap-deficient mutant AH12 in PBS were adjusted to an OD (578 nm) of 1.0 (approximately 10⁹ cells/ml), and 100 μl of the bacterial suspension was added per well. After incubation for 1 h at 37° C. the wells were washed and the number of adherent bacteria was quantified by crystal violet staining at 590 nm.

### Results:

FIG. 2 (A and B) illustrates the contribution of Eap to the adhesion of S. aureus to ICAM-1. In FIG. 2A the adhesion of S. aureus strain Newman and the Newman Eap-deficient mutant strain AH12 to immobilized FBG (filled bars) or ICAM-1 (hatched bars) (each 5 μg/ml) is shown. In FIG. 2B the adhesion of S. aureus strain Newman to immobilized ICAM-1 in the absence or presence of increasing concentrations of EapN is shown. Adhesion is expressed as absorbance at 590 nm and data are mean ±SEM (n=3) of a typical experiment; similar results were obtained in at least three separate experiments.

One consequence of the described direct binding interaction between ICAM-1 and Eap is the possible contribution of Eap in S. aureus adhesion to ICAM-1 on endothelial cells. Although Eap binds to FBG, Eap does not mediate S. aureus adhesion to FBG; here, bacterial adhesion is predominantly dependent on clumping factor. When the adhesion of S. aureus strain Newman and mutant AH12 to FBG was compared, no difference between both strains was observed (FIG. 2A); addition of soluble clumping factor blocked adhesion of both strains to FBG by >50-60% (not shown). On the other hand, S. aureus Newman adhered to ICAM-1 and endothelial cells and this adhesion was markedly reduced in Eap-deficient strain AH12 (FIG. 2A). Moreover, the exogenous addition of Eap dose-dependently inhibited the adhesion of strain Newman to immobilized ICAM-1 (FIG. 2B). These data indicate that Eap secreted from S. aureus and rebound to the bacterial surface plays an important role for the ICAM-1-dependent adhesion of S. aureus to endothelial cells.

The inflammatory situations treated in accordance with the present invention include acute and allergic inflammatory reactions, including responses to radiation, infection, chemicals, allergins, and injury. Examples of specific conditions that can be treated include allergy, asthma, arthritis, psoriasis, skin sunburn, inflammatory pelvic disease, inflammatory bowel disease, urethritis, uvitis, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitis, and cholangititis.

Finally, it is known that non-regulated adhesiveness of leukocytes circulating tumour cells and/or endothelial cells may result in uncontrolled cellular extravasation causing atherosclerosis, rheumatoid arthritis or leading to tumour metastasis. In such pathological processes Eap derived sequences could be devised as ICAM-1 blocking agents to achieve an antiadhesive potential during therapeutic interventions. Therefore, in a further aspect the invention also provides a method of treating a mammal suffering from a cancer, or susceptible of developing a tumour metastasis, e.g. after a cancer therapy including a surgical removal of a tumour, by administering Eap or a suitable peptide portion thereof to said mammal. Accordingly, in relation to this further aspect, Eap or a suitable peptide fraction thereof is used in the manufacture of a medicament to be given as part of a cancer therapy.

The protein or peptide of the invention may be produced by chemical synthesis or by recombinant expression according to conventional methods. For example, the proteins and peptides according to the invention can be obtained by using a host organism transformed or transfected with an expression vector obtained by insertion of a gene according to the invention, or part thereof, into a vector in a conventional manner. The vector which is used to construct the expression vector is not particularly limited, but specific examples include plasmids such as pET (Stratagen) and the like; and phages such as M13 (NEB), phage display libraries and the like. As expression regulatory sequence can among others T7 promotors and lac promotors be used.

An appropriate host to be transformed or transfected with the expression vector can be chosen among for example E. coli, or Bacillus subtilus. The transformed or transfected host is cultured and proliferated under suitable conditions, as known to the person skilled in the art.

After culturing, the peptides of the present invention may be purified by, for example, chromatography, precipitation, and/or density gradient centrifugation.

The purified preparation containing one or several proteins according to the invention, or parts thereof, is then formulated as a pharmaceutical composition, as for example a vaccine, or in a mixture with adjuvants. If desired the proteins are fragmented by standard chemical or enzymatic techniques to produce peptide segments.

The protein or peptide according to the invention can be formulated as pharmaceutical compositions and administered to a mammal subject, e.g. a human patient, in any suitable form, depending on the subject and the specific condition being treated. The compositions may be adapted for local or systemic, oral or parenteral administration, i.e. by intravenous, intramuscular, topical or subcutaneous routes. Administration may be e.g. by inhalation or insufflation, topically, vaginally, rectally, by intracavitary administration, transdermally, intradermally, intraperitoneally or nasally.

By oral administration the protein or peptide compositions may comprise a pharmaceutically acceptable vehicle, such as an inert diluent or an assimilable edible carrier, and any suitable excipient. Any suitable dosage form may be used, such as hard or soft shell gelatin capsules, tablets, buccal compositions, troches, capsules, elixirs, suspensions, syrups, wafers, and the like, or by direct incorporation in the food of the patient's diet.

The tablets and the like also may contain any suitable constituents, e.g. binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose or aspartame, flavouring agents such as peppermint, oil of wintergreen, or cherry flavouring. Additionally, capsules may contain, a liquid carrier, e.g. a vegetable oil. Also, coating materials may be provided, such as gelatin, wax, shellac or sugar and the like. A syrup or elixir may contain the peptide compositions, a sweetening agent, preservatives, such as methyl and propylparabens, and flavourings. Sustained-release preparations and devices, such as sustained release capsules or patches, may also be used.

The protein or peptide compositions according to the invention also may be solutions or dispersions to be administered intravenously or intraperitoneally by infusion or injection. The pharmaceutical dosage forms suitable for injection or infusion can include sterile aqueous solutions or dispersions or sterile powders comprising the protein or peptide composition according to the invention for the extemporaneous preparation of sterile injectable or infusible solutions or dispersions. Also it may be encapsulated in liposomes. Any suitable liquid carrier or vehicle may be used, e.g. water, ethanol, a polyol (for example, glycerol, propylene glycol, liquid polyethylene glycols, and the like), vegetable oils, and mixtures thereof. Other conventional additives are e.g. preservatives.

For topical administration, including also e.g. vaginal, rectal, intracavitary and buccal administration, it generally will be desirable to administer the protein or peptide compositions according to the invention in combination with a dermatologically acceptable solid or liquid carrier, well-known to the man skilled in the art. Liquid compositions can be applied from absorbent pads, used to impregnate bandages and other dressings, or sprayed onto the affected area, and may include any suitable viscosity modifiers or thickeners to form gels, ointments, and the like.

The amount of the protein or peptide composition according to the invention required for use in treatment will vary with the route of administration, the nature of the condition being treated and the age and condition of the patient. In general, however, a suitable dose will be in the range of from about 0.2 mg/kg of body weight to 20 mg/kg of body weight by systemic administration and from about 0.2 mg/kg of body weight to 100 mg/kg body weight by local administration.

The protein or peptide composition according to the invention conveniently may be presented in a single dose or as multiple doses administered at appropriate time intervals over the day.

### REFERENCES

1. Palma, M., A. Haggar, and J.-I. Flock. 1999. Adherence of Staphylococcus aureus is enhanced by an endogenous secreted protein with broad binding activity. J. Bacteriol. 181:2840-2845.
2. McGavin, M. H., D. Krajewska-Pietrasik, C. Ryden, and M. Höök. 1993. Identification of a Staphylococcus aureus extracellular matrix-binding protein with broad specificity. Infect. Immun. 61 (6):2479-2485.
3. Jönsson, K., D. McDevitt, M. H. McGavin, J. M. Patti, and M. Höök. 1995. Staphylococcus aureus expresses a major histocompatibility complex class II analog. J. Biol. Chem. 270 (37):21457-21460.
4. Jahreis, A., Y. Yousif, J. A. Rump, R. Dräger, A. Vogt, H. H. Peter, and M. Schlesier. 1995. Two novel cationic staphylococcal proteins induce IL-2 secretion, proliferation and immunoglobulin synthesis in periferal blood mononuclear cells (PBMC) of both healthy controls and patients with common variable immunodeficiency. Clin. Exp. Immunol. 100:406-411.
5. Fujigaki, Y., Y. Yousif, T. Morioka, S. Batsford, A. Vogt, A. Hishida, and M. Miyasaka. 1998. Glomerular injury induced by cationic 70-kD staphylococcal protein; specific immune response is not involved in early phase in rats. J. Pathol. 184:436-445.
6. Kreikemeyer, B., D. McDevitt, V. Kapur, and M. Hook. 1999. The MHC class II analog protein (Map) expressed by S. aureus: Prevalence of the map gene, expression of size variants and characterization of a second gene class. NCBI database, Accession AJ223806.
7. Yousif, Y., R. Draeger, M. Schiltz, H. Peter, and M. Schleisier. 1997. Nucleotide sequence of a S. aureus gene encoding outer surface binding 70 kD protein. NCBI database, Accession Y10419
8. Hussain M et al., NCBI database Accession AJ132841.
9. Bosse, R. et al. 1994. Eur. J. Immunol. 24: 3019.
10. Borges; E. et al. 1997. Blood 90: 1934.

1. A method of treatment of an inflammatory condition related to an autoimmune disease, comprising
administering to a mammal in need of such treatment an amount effective for such treatment of an anti-inflammatory drug comprising (i) a protein or (ii) a peptide fraction of said protein comprising at least one repeating unit thereof, said protein being a protein designated Eap (Extracellular adherence protein).

2. The method of claim 1, wherein the protein is SEQ ID NO: 1.

4. The method of claim 1, wherein the mammal is a human.

5. The method of claim 1, wherein the autoimmune disease is asthma.

6. The method of claim 1, wherein the autoimmune disease is arthritis.

7. The method of claim 1, wherein the autoimmune disease is psoriasis.

8. The method of claim 1, wherein the autoimmune disease is inflammatory bowel disease.

9. The method of claim 1, wherein the autoimmune disease is rheumatoid arthritis.

10. A method of treatment of an inflammatory condition related to an autoimmune disease, comprising
administering to a mammal in need of such treatment an amount effective for such treatment of an anti-inflammatory drug preventing recruitment of leukocytes or neutrophils to a site of inflammation, said drug comprising (i) a protein or (ii) a peptide fraction of said protein comprising at least one repeating unit thereof, said protein being a protein designated Eap (Extracellular adherence protein).

11. The method of claim 10, wherein the protein is SEQ ID NO: 1.

12. The method of claim 10, wherein the mammal is a human.

13. The method of claim 10, wherein the autoimmune disease is asthma.

14. The method of claim 10, wherein the autoimmune disease is arthritis.

15. The method of claim 10, wherein the autoimmune disease is psoriasis.

16. The method of claim 10, wherein the autoimmune disease is inflammatory bowel disease.

17. The method of claim 10, wherein the autoimmune disease is rheumatoid arthritis.
